(19) United States
(12) Patent Application Publication
(10) Pub. No.: US 2011/0015575 A1
(43) Pub. Date: Jan. 20, 2011

(54) SPINAL NEEDLE LIGHT GUIDE APPARATUS AND METHOD OF DELIVERY

(76) Inventor: Thomas Perez, Chicago, IL (US)

Correspondence Address: ADAM K. SACHAROFF;MUCH SHELIST DENENBERG AMENT & RUBENSTEIN, 191 N. WACKER DRIVE, Suite 1800, CHICAGO, IL, 60606-1615, US

(21) Appl. No.: 12/503,611

(22) Filed: Jul. 15, 2009

(51) Int. Cl. A61M 5/178; A61F 11/00; A61H 1/00

(52) U.S. Cl. 604/164.01; 606/108; 601/15

(57) ABSTRACT

In one embodiment there is provided a spinal needle having a cannula defining an interior hollowed region with a longitudinal length and opposite proximal and distal ends. The cannula includes an opening at its distal end. The spinal needle further includes a UV light source and a light transmission tube positioned within the cannula for transmitting UV light emitting from the UV light source. The light transmission tube having a length extending through the longitudinal length of the cannula and having a distal end positioned at or about the distal end of the cannula.

### BACKGROUND OF THE INVENTION

1. Field of the Invention

The present invention relates to a method and apparatus for providing light to cerebrospinal fluid.

2. Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic. In these circumstances, the UV light is applied either to the patient's skin or directly to the blood. If the UV light is applied to the skin it is typically provided to the patient's skin either with a wrap or lamp. Applying the UV light directly to a patient's blood supply is known as photoluminescence or UV blood illumination (UBI). UV blood illumination increases oxygen, destroys toxins and boosts the immune system.

However, diseases which are caused or relate to viruses in the brain, which may even include multiple sclerosis, are not treatable through UV blood illumination. The ability to treat these infections, viruses, and bacterial in the brain may require UV illumination of the cerebrospinal fluid also referred to as the cerebral spinal fluid or CSF (the terms are used interchangeable herein) that surrounds the brain and spinal cord. Since the cerebrospinal fluid surrounds/travels around the brain, the UV illumination of the CSF may treat infections, viruses, and bacterial in the brain.

There is thus a need for a method of providing UV light to the cerebrospinal fluid. In accordance with the method an apparatus has been developed.

### SUMMARY OF THE INVENTION

In one embodiment of the present invention there is provided a novel spinal needle used to deliver UV light to the cerebrospinal fluid in the subarachnoid space. The spinal needle includes a cannula defining an interior hollowed region having a longitudinal length with opposite proximal and distal ends and an opening at its distal end. The needle includes a UV light source, and a light transmission tube positioned within the cannula for transmitting UV light emitting from the UV light source. The light transmission tube has a length extending through the longitudinal length of the cannula and having a distal end positioned at or about the distal end of the cannula.

In using the spinal needle, the operator pierces the epidermis of the body cavity and advances the cannula toward the body cavity until the distal end of the cannula enters the subarachnoid space. The UV light source may be activated such that the UV light will emit from the end of the light transmission tube to radiate cerebrospinal fluid in the subarachnoid space.

In other embodiments, the light transmission tube is a liquid light guide or one or more fiber optic strands. In other aspects of the embodiments, the light transmission tube includes a lens positioned about the distal end defined thereby. The light tube may also have an end with a connector for attaching the light tube to the light transmission tube.

In yet other aspects and embodiments, the spinal needle may include a catheter positioned within the light tube and having a length extending through the longitudinal length of the cannula and substantially positioned against the light transmission tube with a distal end positioned about the end of the distal end of the cannula. The catheter may be used to delivery medicine or drugs to help reduce the effects of a spinal headache.

In other aspects, the spinal needle includes a primary tube having an end with a connector for attaching the primary tube to a base connector defined at an end defined by the cannula for transmitting the UV light from a UV light source through the primary tube to the light transmission tube. In this aspect, the catheter can be positioned within the primary tube, and along the length of the cannula and substantially positioned against the light transmission tube and having a distal end positioned about the end of the distal end of the cannula.

Numerous advantages and features of the invention will become readily apparent from the following detailed description of the invention and the embodiments thereof, and from the accompanying drawings.

### BRIEF DESCRIPTION OF THE DRAWINGS

A fuller understanding of the foregoing may be had by reference to the accompanying drawings, wherein:

FIG. 1 is a prior art illustration of spinal needles currently used and the method for administering medication to an epidural space and the improper puncturing of the subarachnoid space;

FIG. 2A is a partial sectional view of a spinal needle in accordance with one or more of the embodiments presented herein to treat cerebrospinal fluid with a UV light;

FIG. 2B is a partial exploded view of the spinal needle showing the cannula and the light guide;

FIG. 2C is a perspective view of the spinal needle from FIG. 2A; and

FIG. 3 is an illustration of a spinal needle in accordance with one or more of the embodiments presented herein treating the cerebrospinal fluid with a UV light.

### DETAILED DESCRIPTION OF THE INVENTION

While the invention is susceptible to embodiments in many different forms, there are shown in the drawings and will be described herein, in detail, the preferred embodiments of the present invention. It should be understood, however, that the present disclosure is to be considered an exemplification of the principles of the invention and is not intended to limit the spirit or scope of the claims by the embodiments illustrated.

Light at one or more therapeutic wavelength, such as ultraviolet light (UV), is used to treat many diseases including infections, poisoning, fatigue, allergies, hepatitis, cancer and HIV. UV light increases the oxygen combining power, destroys toxins, viruses, fungi, bacteria, and boosts the immune system. UV light also sterilizes the blood and acts as an antibiotic. It is now proposed to use the UV light to treat the cerebrospinal fluid that occupies the subarachnoid space in a person's spinal region. Preferably, UV light at one or more therapeutic wavelength is utilized in the present invention. More preferably the light is either UV-A or UV-C light is utilized in the present invention. For some conditions and/or diseases UV-A light is more effective than UV-C and for other conditions and/or diseases UV-C light is more effective than UV-A light. The wavelengths or wavelengths of light to be used to treat the patient are selected based on the wavelength or wavelength that will best treat the condition or disease of the patient.

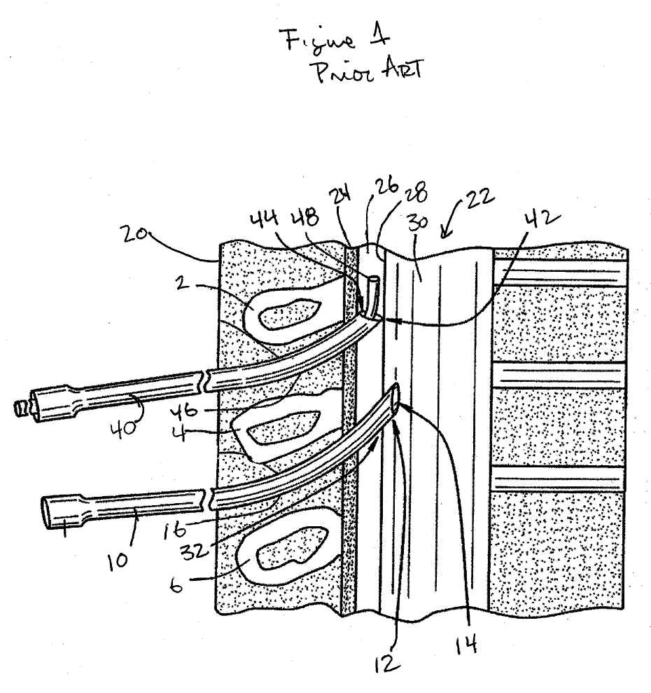

Referring now to prior art FIG. 1, as is well known in the field of spinal needle usage administering spinal anesthetic is preferably done in such a manner to prevent the development of post dural puncture headache. Headaches are commonly associated when the subarachnoid space is punctured and cerebrospinal fluid from the subarachnoid space leaks through the puncture site into the epidural compartment of the spine. As illustrated, a first spinal needle 10 is shown inserted between the second 4 and the third 6 lumbar vertebrae located in the back. The distal end 12 of the first spinal needle 10 includes a beveled end 14 which helps cut through the various layer of tissue. In the example, the first spinal needle 10 is shown passing through the body tissue 20 toward the spine 22. The distal end 12 of the first spinal needle 10, forming an end of the cannula 16, passes through the body tissue 20 and the layer of yellow ligament 24 surrounding the spine. The first spinal needle 10 passes through the epidural space 26 and accidentally penetrates 32 the dura mater 28 to enter into the subarachnoid space 30. As mentioned, in this example cerebrospinal fluid leaks from the subarachnoid space 30 into the epidural space 26 causing a spinal headache. Continuing to refer to FIG. 1, in another example of the prior art, a second needle 40 is shown having a distal end 42 with a beveled end 44 near the cannula 46 is properly inserted into the epidural space 26. The second needle 40 is further illustrated as having a catheter 48 for the delivery of a liquid spinal anesthetic.

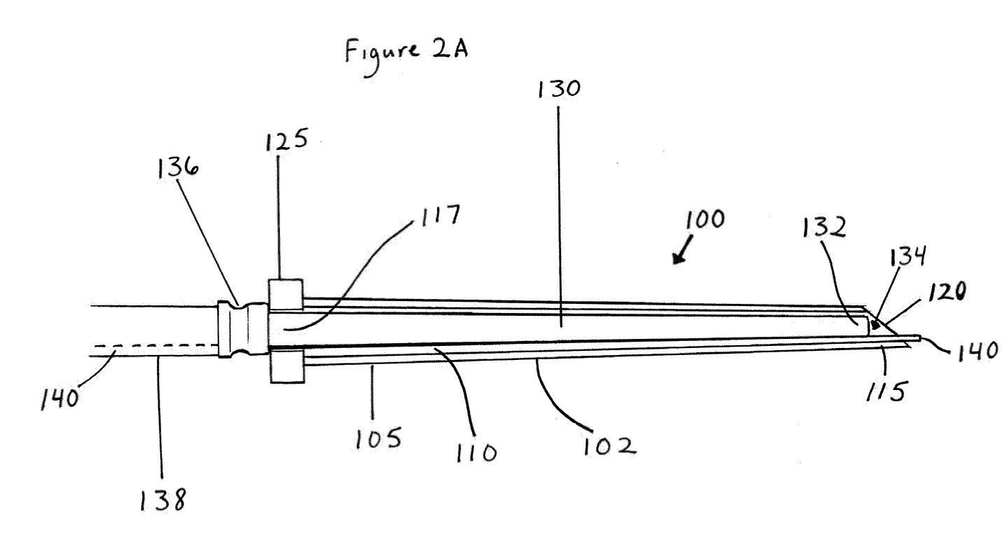

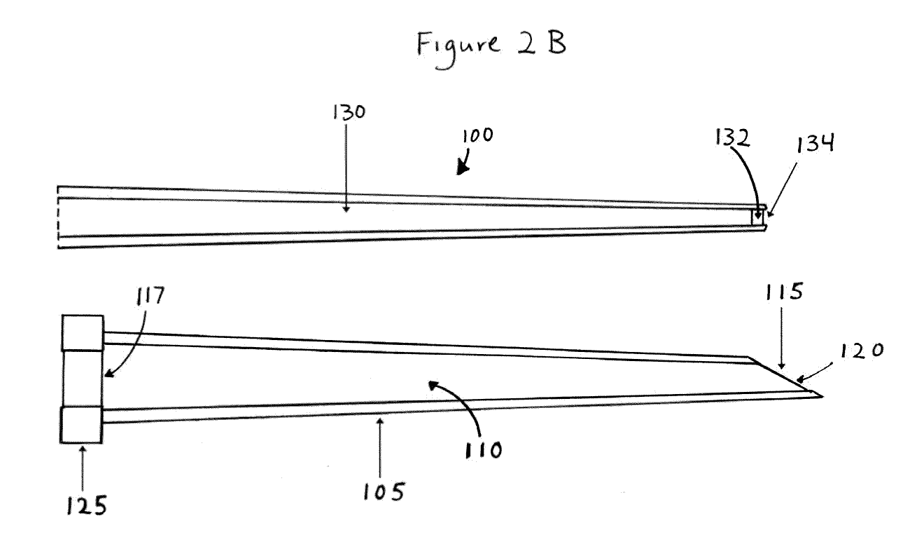

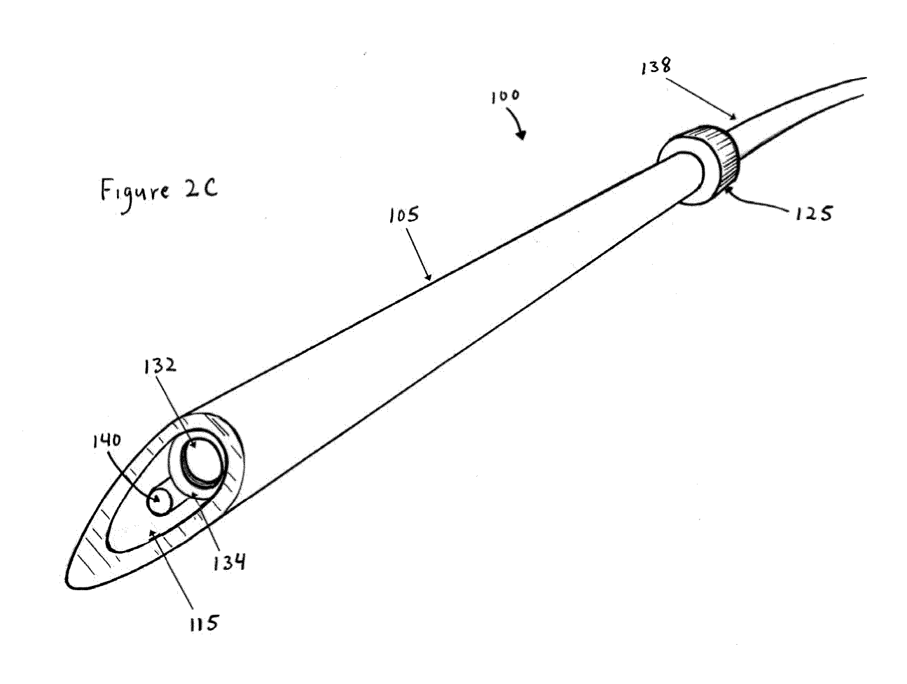

Referring now to FIGS. 2a through 2c, to accordance to the embodiments presented herein, there is provided a spinal needle 100 developed to accommodate the treatment aspects of the present invention. The spinal needle 100 may be of a gauge equal to or less than 29. The gauge of the spinal needle 100 may be determined by the use or administrators preference. The spinal needle 100 includes an cannula 102 with an outer casing 105 defining a hollowed interior 110 and includes a distal end 115 with a beveled end 120 used to penetrate the body tissue and dura layers. The cannula 102 further includes a needle base 125.

The hollowed interior 110 of the cannula 102 receives a light transmission tube 130. The light tube 130 may be a liquid light guide or fiber optic cable, or other similar device, including a UV light emitting bulb positioned at an end 132 adjacent or near the distal end 115 with power transmission cables extending through the light transmission tube 130 or a UV light emitting bulb positioned at a proximal end 117 of the hollowed interior 110 with a light transmission cable (such as a liquid light guide of fiber optic strands) extending through the light tube 130. The important aspect of the light tube is that UV light emits from the end 132 near the distal end 115 of the cannula 102.

The light source (not shown) may be LEDs or a light guide transferring the light from a source bulb, medical grade UV light bulb, or similar source that emits UV light at a therapeutic wavelength(s). More preferably, the light is UV-C light, UV-A, UV-B light or a combination thereof. Alternatively, a plurality of LEDs, having one or more different wavelengths of light at one or more therapeutic wavelengths is used. It is preferable that a substantial portion of the emitted light be UV-C and/or UV-A. There may alternatively be fused silica fiber optic bundles in the tube 130. Fused silica fiber optic bundles may be used because they do not emit any heat. The fiber optic bundles deliver UV light at a therapeutic wavelength sublingually.

Positioned at the end 132 of the light tube 130 is a lens 134 to help protect the light tube and help disperse and emit the UV light. The UV light is powered by a power source not shown but may be well known sources such as batteries or AC power with if necessary DC converter to power the UV light source.

It may be preferably designed to have the light tube 130 removably and slidably positioned within the hollowed interior 110 of the cannula 102. To help ensure the light tube 130 is secured within the cannula 102, the light tube 130 includes a connector 136 which secures to the needle base 125. A base tube 138 would extend past the connector 136 to secure to a power source, light source, or other equipment needed to facilitate proper use of the spinal needle 100.

In addition, the base tube 138 may secure to equipment for the delivery of medication, in this instance the base tube 138 would include a separate interior channel, interior tubing, or a catheter, generally referenced as catheter 140 for the delivery of medication that would be separate from the light tube 130. The medication could be designed to help reduce the spinal headache or other medications used for the treatment.

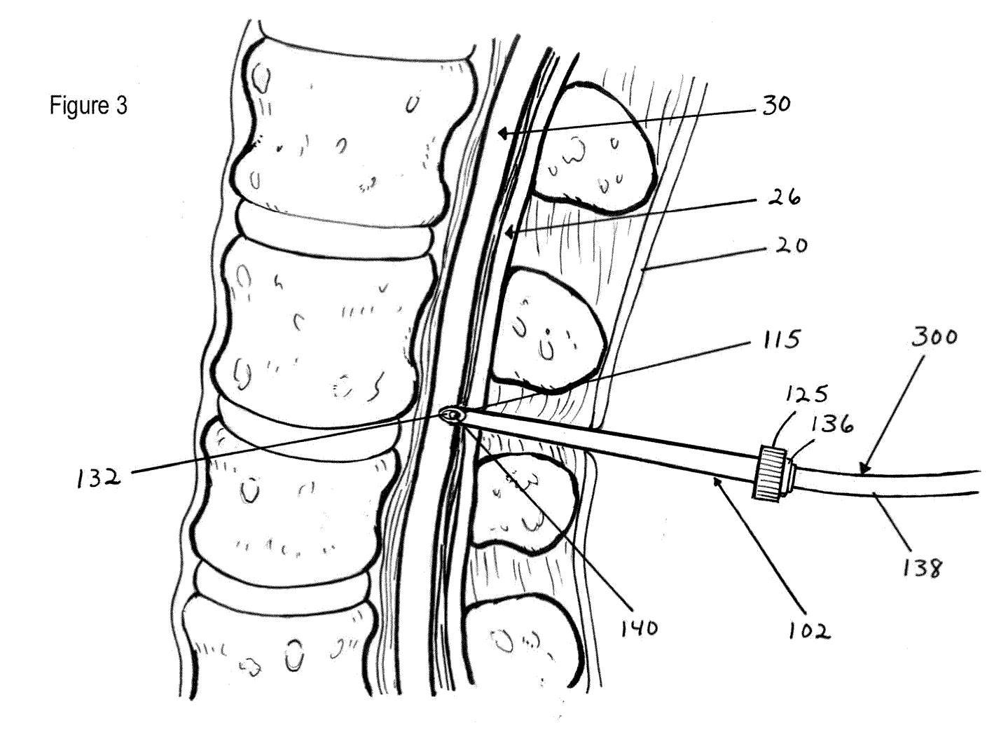

Referring now also to FIG. 3, during treatment the spinal needle of the present invention 300 is positioned through the body tissue 20 between two lumbar vertebra through the epidural space 26 and penetrating the dura mater 28 to enter into the subarachnoid space 30. Once the distal end 115 of the cannula 102 or the spinal needle tip enters the subarachnoid space 30, the UV light source can be activated such that the UV light is emitted outwardly from the spinal needle tip and can radiate cerebrospinal fluid in the subarachnoid space. In addition, medication may be administered through the catheter 140 if desired. The duration of the treatment may be determined by the physician.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred.

I claim:

1. A spinal needle, comprising of a cannula defining an interior hollowed region having a longitudinal length with opposite proximal and distal ends and an opening at its distal end, the improvement comprising:
a UV light source, and
a light transmission tube positioned within the cannula for transmitting UV light emitting from the UV light source, the light transmission tube having a length extending through the longitudinal length of the cannula and having a distal end positioned at or about the distal end of the cannula.

2. The needle of claim 1, wherein the light transmission tube is a liquid light guide or one or more fiber optic strands.

3. The needle of claim 1 further comprising a light tube having an end with a connector for attaching the light tube to the light transmission tube.

4. The needle of claim 3 further comprising a catheter positioned within the light tube, the catherter having a length extending through the longitudinal length of the cannula and substantially positioned against the light transmission tube and having a distal end positioned about the end of the distal end of the cannula.

5. The needle of claim 1 wherein the light transmission tube includes a lens positioned about the distal end defined thereby.

6. A spinal needle, comprising of a cannula defining an interior hollowed region having a longitudinal length with opposite proximal and distal ends and an opening at its distal end, the improvement comprising:
a UV light source, and
a light transmission tube positioned within the cannula for transmitting UV light emitting from the UV light source, the light transmission tube having a length extending through the longitudinal length of the cannula and having a distal end positioned at or about the distal end of the cannula, and the light transmission tube includes a lens positioned about the distal end defined thereby.

7. The needle of claim 6, wherein the light transmission tube is a liquid light guide or one or more fiber optic strands.

8. The needle of claim 6 further comprising a light tube having an end with a connector for attaching the light tube to the light transmission tube.

9. The needle of claim 6 further comprising a catheter positioned within the light tube, the catherter having a length extending through the longitudinal length of the cannula and substantially positioned against the light transmission tube and having a distal end positioned about the end of the distal end of the cannula.

10. A method of UV light delivery to cerebrospinal fluid in a subarachnoid space for a body cavity, said method comprising:
providing a hollow cannula with a distal end;
piercing epidermis of the body cavity and advancing the cannula toward the body cavity until the distal end of the cannula enters the subarachnoid;
providing UV light source from a UV light source through a light transmission tube positioned within the cannula, the light transmission tube having an end positioned about the distal end of the cannula;
emitting UV light from the end of the light transmission tube to radiate cerebrospinal fluid in the subarachnoid space.

11. The method of claim 10, wherein the light transmission tube is a liquid light guide or one or more fiber optic strands.

12. The method of claim 10 further comprising:
providing a primary tube having an end with a connector for attaching the primary tube to a base connector defined at an end defined by the cannula for transmitting the UV light from a UV light source through the primary tube to the light transmission tube.

13. The needle of claim 12 further comprising:
providing a catheter positioned within the primary tube, the catherter having a length extending through the longitudinal length of the cannula and substantially positioned against the light transmission tube and having a distal end positioned about the end of the distal end of the cannula.

14. The needle of claim 10, wherein the light transmission tube includes a lens positioned about the distal end defined thereby.
